(19) United States
(12) Patent Application Publication
(10) Pub. No.: US 2025/0366827 A1
(43) Pub. Date: Dec. 4, 2025

(54) ULTRASOUND IMAGING DEVICE AND METHOD FOR PRESENTING LESION DISTRIBUTION

(71) Applicant: SHENZHEN MINDRAY BIO-MEDICAL ELECTRONICS CO., LTD., Shenzhen (CN)

(72) Inventors: Wenhui CHEN, Shenzhen (CN); Wenjing JIA, Shenzhen (CN); Yaoxian ZOU, Shenzhen (CN); Muqing LIN, Shenzhen (CN)

(73) Assignee: SHENZHEN MINDRAY BIO-MEDICAL ELECTRONICS CO., LTD., Shenzhen (CN)

(21) Appl. No.: 19/226,195

(22) Filed: Jun. 3, 2025

(30) Foreign Application Priority Data
Jun. 3, 2024 (CN) 202410713489.0

(51) Int. Cl. A61B 8/00; A61B 8/08; G06T 7/00; G06T 11/20

(52) U.S. Cl. CPC A61B 8/461; A61B 8/085; G06T 7/0012; G06T 11/206; G06T 2200/24; G06T 2207/10132; G06T 2207/20036; G06T 2207/30096

(57) ABSTRACT

Disclosed are ultrasound imaging devices and methods for presenting lesion distribution, which may include: acquiring a target tissue diagram of a target section of a target tissue; displaying the target tissue diagram as the background of a lesion distribution diagram on a display interface to provide a reference for a user to edit a lesion diagram in the lesion distribution diagram; in response to a user editing operation, determining a morphological parameter comprising at least one of appearance, dimension, orientation, and relative position to the target tissue diagram; and displaying the lesion diagram on a corresponding region of the target tissue diagram according to the morphological parameter.

### CROSS-REFERENCE TO RELATED APPLICATION

This application claims priority to and the benefit of Chinese Patent Application No. 202410713489.0, filed on Jun. 3, 2024, the disclosure of which is incorporated herein by reference in its entirety.

### TECHNICAL FIELD

The present disclosure relates to medical devices, in particularly to ultrasound imaging devices and methods for presenting lesion distribution.

### BACKGROUND

Ultrasound, as the most commonly used and reliable first-line clinical modality for assessing lesions, assists doctors in achieving accurate diagnoses. Current examination methods typically involve sector scanning observation of lesions in 2D mode, followed by textual diagnostic conclusions provided in ultrasound reports for clinical reference. However, existing ultrasound reports present interpretation challenges for clinicians, as they must rely on clinical experience and mental reconstruction, often necessitating further consultation with sonographers, which is time-consuming and labor-intensive. The following explanation uses uterine lesions as an example.

The uterus, as one of the most critical organs in the female reproductive system, is essential for maintaining hormone secretion and normal fertility. Anatomically divided into the uterine cervix, endometrium, myometrium, and uterine wall, the uterus has seen a gradual increase in various pathological conditions due to changes in living environments and lifestyles associated with industrialized societies, thereby garnering increasing attention. For instance, uterine fibroids—the most prevalent gynecological tumor-like disease-affect 70-80% of women of childbearing age, predominantly those aged 30-50. These lesions may be accompanied by symptoms such as bleeding, pain, and infertility, posing significant impacts on women's physical and mental health. Therefore, achieving timely detection and accurate diagnosis of uterine lesions is of paramount importance.

Clinicians require precise localization of uterine lesion distribution (including intrauterine location, quantity, and dimensions of lesions), classification, and dynamic changes to develop appropriate treatment plans uterine fibroids as an example, patients often exhibit a strong desire for fertility with high demand for fertility preservation, while also prioritizing preservation of the uterus as a female symbol. Early clinical intervention and treatment are necessary to mitigate malignancy risks. Treatment strategies are primarily formulated based on the distribution characteristics of uterine fibroids (location, classification, quantity, dimension) and patient preferences. Consequently, rapid and precise acquisition of uterine fibroid distribution data is critically important for clinicians.

In accordance with clinical guidelines and practices, ultrasound serves as the most prevalent and reliable first-line modality for evaluating uterine fibroids, offering advantages of high diagnostic sensitivity and specificity to accurately assess fibroid distribution and classification, thereby demonstrating significant clinical value. Comprehensive preoperative evaluation of fibroid distribution enables clinicians to select optimal surgical approaches, reduce operative trauma and postoperative complications, effectively shorten recovery time, and better preserve fertility. Concurrently, it facilitates longitudinal monitoring of disease progression. However, current ultrasound assessment results are communicated solely through textual descriptions in reports, which present interpretative challenges for clinicians. Gynecologists predominantly rely on mental reconstruction combined with verbal consultations with sonographers to obtain fibroid distribution information-a process that is time-consuming and labor-intensive, ultimately compromising precision in clinical decision-making.

Therefore, the transmission efficiency of patients' lesion distribution information between sonographers and clinicians requires improvement and enhancement.

### SUMMARY

In an embodiment, a method for presenting lesion distribution is provided, which may include:
acquiring a target tissue diagram of at least one target section of a target tissue, where the target tissue diagram of the target section is a schematic diagram for presenting a morphology of the target tissue having at least one lesion on the target section;
displaying the target tissue diagram of the target section on a display interface;
determining a morphological parameter of a lesion diagram of a lesion in response to an editing operation, the morphological parameter including at least one of an appearance of the lesion diagram, a dimension of the lesion diagram, an orientation of the lesion diagram, and a relative position between the lesion diagram and the target tissue diagram; and
displaying the lesion diagram on a corresponding region of the target tissue diagram according to the morphological parameter of the lesion diagram, thereby obtaining the lesion distribution diagram of the target section.

In an embodiment, acquiring the target tissue diagram of at least one target section of the target tissue may include:
obtaining an ultrasound image of the at least one target section of the target tissue; and
generating the target tissue diagram of the target section based on the ultrasound image of the target section.

In an embodiment, the target tissue diagram of the target section may include a contour of an anatomical structure of the target tissue, and the method may further include:
receiving a spatial relationship between the lesion and the anatomical structure determined by a user;
when the spatial relationship exhibits overlap, performing a first differentiated display on an overlapping portion of the contour of the anatomical structure with the lesion diagram to indicate that the spatial relationship between the lesion and the anatomical structure is overlapping; and
when the spatial relationship exhibits compression, performing a second differentiated display on the overlapping portion of the contour of the anatomical structure with the lesion diagram to indicate that the spatial relationship between the lesion and the anatomical structure is compressive; where the first differentiated display is different from the second differentiated display.

In an embodiment, the method may further include:
when the determined morphological parameter comprises the relative position between the lesion diagram and the target tissue diagram, generating a lesion stalk diagram based on the relative position between the lesion diagram and the target tissue diagram, and displaying the lesion stalk diagram between the lesion diagram and the target tissue diagram; or
receiving a user-initiated operation for drawing a lesion stalk diagram between the lesion diagram and the target tissue diagram, generating the lesion stalk diagram in response to the operation, and displaying the lesion stalk diagram between the lesion diagram and the target tissue diagram;
where the lesion stalk diagram has a first end connected to the lesion diagram and a second end connected to the target tissue diagram.

In an embodiment, the target tissue diagram of the target section may include a contour of the anatomical structure of the target tissue, and
the second end of the lesion stalk diagram is connected to a connection point on the target tissue diagram located at a closest point on the contour of the anatomical structure adjacent to the lesion diagram, where a position of the connection point is adjustable on the lesion distribution diagram.

In an embodiment, generating the lesion stalk diagram based on the relative position between the lesion diagram and the target tissue diagram may include:
when the lesion diagram is located outside the target tissue diagram, or when the lesion diagram is located in a cavity within the target tissue diagram, generating the lesion stalk diagram.

In an embodiment, the editing operation may include an appearance selection operation and a region selection operation; and
said determining a morphological parameter of the lesion diagram in response to an editing operation, and displaying the lesion diagram on a corresponding region of the target tissue diagram according to the morphological parameter of the lesion diagram may include:
displaying a plurality of lesion diagrams with different appearances on the display interface for user selection, where the appearances represent at least one of a lesion type being benign or malignant, and a lesion contour;
taking a user-selected lesion diagram as the lesion diagram in the lesion distribution diagram in response to the appearance selection operation; and
displaying the lesion diagram in a user-selected region in response to the region selection operation.

In an embodiment, the method may further include:
acquiring a dimensional measurement of the lesion on an ultrasound image of the lesion; and
determining the dimension of the lesion diagram based on the dimensional measurement.

In an embodiment, the dimensional measurement may include at least one of a measured value of a length of the lesion, a measured value of a width of the lesion and a measured value of a height of the lesion; and
the plurality of lesion diagrams with different appearances may include: a lesion diagram with an appearance representing a benign lesion, and a plurality of lesion diagrams with appearances respectively representing varying malignancy levels.

In an embodiment, the method may further include:
deleting a displayed lesion diagram in response to a user-initiated deletion operation on a selected lesion diagram; or
adjusting the morphological parameter of the lesion diagram in response to the user editing operation, and updating the displayed lesion diagram according to the adjusted morphological parameter.

In an embodiment, said adjusting the morphological parameter of the lesion diagram in response to the user editing operation, and updating the displayed lesion diagram according to the adjusted morphological parameter may include:
in response to a user-initiated operation for moving the displayed lesion diagram, moving the displayed lesion diagram so as to adjust the relative position between the lesion diagram and the target tissue diagram; or
in response to a user-initiated operation for stretching or scaling the displayed lesion diagram, stretching or scaling the displayed lesion diagram so as to adjust the appearance and/or dimension of the lesion diagram; or
in response to a user-initiated operation for rotating the displayed lesion diagram, rotating the displayed lesion diagram so as to adjust the orientation of the lesion diagram.

In an embodiment, the display interface further displays a tumor classification guide diagram configured to prompt a user to determine a tumor type of the lesion, where the tumor classification guide diagram includes a plurality of schematic graphics of different tumor types, each schematic graphic having a feature indicative of a tumor type, and different tumor types exhibiting different features; and the method may further include:
determining the tumor type of the lesion in response to a user-initiated classification operation, and displaying a feature corresponding to the determined tumor type on the lesion diagram; or
when the determined morphological parameter includes the relative position between the lesion diagram and the target tissue diagram, determining the tumor type of the lesion according to a predetermined classification rule based on the relative position, and displaying a feature corresponding to the determined tumor type on the lesion diagram.

In an embodiment, the method may further include:
based on a type of the target section, displaying, on the display interface, a patient orientation corresponding to the lesion distribution diagram of the target section; where, the type of the target section is one of a sagittal plane, a transverse plane, and a coronal plane, and the patient orientation comprises at least one of left, right, ventral, dorsal, cranial, and caudal orientations.

In an embodiment, said generating the target tissue diagram of the target section based on the ultrasound image of the target section may include:
segmenting a contour of an anatomical structure of the target tissue from the ultrasound image of the target section, and generating the target tissue diagram of the target section based on the segmented contour of the anatomical structure of the target tissue; or
displaying a plurality of predetermined basic tissue structure diagrams of the target section on the display interface for user selection; displaying the ultrasound image of the target section on the display interface for a user to input a plurality of key points on the ultrasound image, the plurality of key points being configured to identify a position and a dimension of an anatomical structure of the target tissue diagram; and mapping positional relationships between the plurality of input key points to a user-selected basic tissue structure diagram to generate the target tissue diagram of the target section; or
displaying a plurality of predetermined basic tissue structure diagrams of the target section on the display interface for user selection; acquiring a dimensional measurement of an anatomical structure of the target tissue from the ultrasound image of the target section; and mapping the dimensional measurement of the anatomical structure to a user-selected basic tissue structure diagram to generate the target tissue diagram of the target section; or
displaying a plurality of predetermined basic tissue structure diagrams of the target section on the display interface for user selection; and taking a user-selected basic tissue structure diagram as the target tissue diagram of the target section.

In an embodiment, the target tissue diagram of the target section and the ultrasound image of the target section are in a substantially 1:1 proportion.

In an embodiment, the target tissue is a uterus, and the anatomical structure of the target tissue comprises a uterine body, an endometrium, and a cervix.

In an embodiment, the ultrasound image of the target section and the lesion distribution diagram are each two-dimensional images.

In an embodiment, an ultrasound imaging device is provided, which may include: an ultrasound probe, a transmitting and receiving circuit, a processor, and a human-machine interface device; where:
the ultrasound probe is configured to transmit ultrasound waves toward a target tissue and receive corresponding ultrasound echoes;
the transmitting and receiving circuit is configured to control the probe to transmit ultrasound waves and receive ultrasound echoes; and
the processor is configured to:
acquire a target tissue diagram of at least one target section of a target tissue, where the target tissue diagram of the target section is a schematic diagram for presenting a morphology of the target tissue having at least one lesion on the target section;
display the target tissue diagram of the target section on a display interface;
determine a morphological parameter of a lesion diagram of a lesion in response to an editing operation, the morphological parameter including at least one of an appearance of the lesion diagram, a dimension of the lesion diagram, an orientation of the lesion diagram, and a relative position between the lesion diagram and the target tissue diagram; and
display the lesion diagram on a corresponding region of the target tissue diagram according to the morphological parameter of the lesion diagram, thereby obtaining the lesion distribution diagram of the target section.

In an embodiment, a computer-readable storage medium is provided, which may include: a computer program stored thereon, the computer program being executable by a processor to:
acquire a target tissue diagram of at least one target section of a target tissue, where the target tissue diagram of the target section is a schematic diagram for presenting a morphology of the target tissue having at least one lesion on the target section;
display the target tissue diagram of the target section on a display interface;
determine a morphological parameter of a lesion diagram of a lesion in response to an editing operation, the morphological parameter comprising at least one of an appearance of the lesion diagram, a dimension of the lesion diagram, an orientation of the lesion diagram, and a relative position between the lesion diagram and the target tissue diagram; and
display the lesion diagram on a corresponding region of the target tissue diagram according to the morphological parameter of the lesion diagram, thereby obtaining the lesion distribution diagram of the target section.

Based on the ultrasound imaging device and the method for presenting lesion distribution in aforementioned embodiments, the target tissue diagram of the target section of the target tissue is acquired; the target tissue diagram is displayed as the background of the lesion distribution diagram on the display interface, providing a reference for a user to edit a lesion diagram in the lesion distribution diagram; the morphological parameter of the lesion diagram is determined in response to the user editing operation, said morphological parameter comprising at least one of: an appearance, a dimension, an orientation, and a relative position between the lesion diagram and the target tissue diagram; and the lesion diagram is displayed on the corresponding region of the target tissue diagram according to the morphological parameter of the lesion diagram, thereby obtaining the lesion distribution diagram of the target section. It is apparent that through editing the lesion diagrams, users can obtain lesion distribution diagrams to present the distribution of lesions in patients, which is easier to understand and grasp compared to ultrasound images, thereby enhancing doctor's work efficiency.

### BRIEF DESCRIPTION OF THE DRAWINGS

FIG. 1 is a structural block diagram of a terminal device provided in some embodiments of the present disclosure;

FIG. 2 is a flowchart of a method for presenting lesion distribution provided in some embodiments of the present disclosure;

FIG. 3 is a flowchart of step 1 in some embodiments;

FIG. 4 is a structural block diagram of an ultrasound imaging device provided in some embodiments of the present disclosure;

FIG. 5 is a schematic diagram illustrating measurements performed on the ultrasound image;

FIG. 6 schematically illustrates a target tissue diagram provided in some embodiments of the present disclosure;

FIG. 7 schematically illustrates a lesion distribution diagram provided in some embodiments of the present disclosure;

FIG. 8 is a schematic diagram of a display interface provided in some embodiments of the present disclosure;

FIG. 9 is a schematic diagram illustrating editing performed on a lesion diagram;

FIG. 10 is a flowchart for presenting a spatial relationship between a lesion and a anatomical structure in the method for presenting lesion distribution;

FIG. 11 schematically illustrates a lesion distribution diagram provided in other embodiments of the present disclosure; and

FIG. 12 schematically illustrates a tumor classification guide diagram in some embodiments of the present disclosure.

### DETAILED DESCRIPTION

Specific embodiments of the present disclosure will now be described in detail with reference to the accompanying drawings. Similar or related components in different embodiments are labeled with associated reference numerals. The following embodiments include detailed descriptions to facilitate understanding of the present disclosure. However, those skilled in the art will readily recognize that certain features may be omitted under specific circumstances or substituted by other components, materials, or methods. In some instances, certain operations related to the present disclosure are not explicitly described or illustrated herein. This intentional exclusion is intentional to avoid obscuring the core technical solutions of the present disclosure. For those skilled in the art, a complete understanding of these operations can be attained through the descriptions provided in this specification and general technical knowledge in the art.

Additionally, the features, operations, or characteristics described in the specification may be combined in any suitable manner to form various embodiments. Similarly, steps or actions in the method descriptions may be reordered or modified in ways that would be obvious to those skilled in the art. Therefore, the sequences presented in the specification and drawings are intended solely to clarify the description of specific embodiments and do not imply mandatory orderings, unless explicitly stated that a particular sequence is required.

The numerical designations assigned to components in this specification, such as ‘first,’ ‘second,’ or similar ordinal terms, serve solely to distinguish described objects and carry no inherent sequential or technical implications. Furthermore, the terms ‘connected’ and ‘coupled’ as used herein encompass both direct and indirect connection (coupling), unless explicitly stated otherwise.

In the present disclosure, after a doctor completes ultrasound scan, a schematic diagram of the tissue structure can be generated based on an ultrasound image, lesion annotation can be performed on the schematic diagram to generate a lesion distribution diagram that reflects the distribution of lesions in the tissue. This visualization enables clinicians to more intuitively and accurately comprehend the distribution of lesions, thereby providing a decision-making foundation for subsequent clinical treatment plans. Below are some embodiments for detailed explanation.

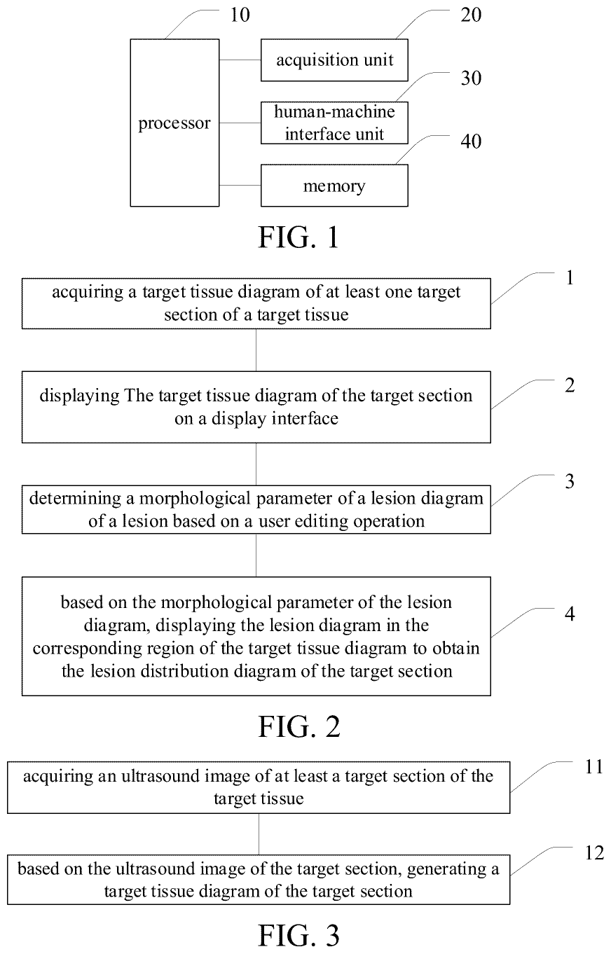

The present disclosure provides a terminal device capable of generating and displaying a lesion distribution diagram in response to a user-initiated operation. As shown in FIG. 1, the terminal device may include a processor 10, an acquisition unit 20, a human-machine interface (HMI) unit 30, and a memory 40. A specific process for generating and displaying the lesion distribution diagram by the terminal device is illustrated in FIG. 2, which may include the following steps:

Step 1: acquiring a target tissue diagram of at least a target section of a target tissue.

For example, the processor 10 acquires the target tissue diagram of at least a target section of a target tissue via the acquisition unit 20. Clinicians may typically require accurate understanding of the location of lesions in tissues to facilitate subsequent examinations and surgeries. Therefore, multiple (two or more) target tissue diagrams of different target sections are obtained in the shown embodiment, enabling comprehensive analysis of lesion distribution from various perspectives.

The target tissue diagrams may be sourced from external devices; for example, external devices stores target tissue diagrams of target sections. The acquisition unit 20 may be a communication unit configured to obtain the target tissue diagrams of at least a target section of the target tissue from an external device via wired or wireless means.

The target tissue diagrams may also be generated automatically by the terminal device itself, for example, as shown in FIG. 3, step 1 may include:

Step 11: acquiring an ultrasound image of at least a target section of the target tissue by the processor 10 via the acquisition unit 20.

Similarly, the acquisition unit 20 may obtain the ultrasound image from an external device or generate the ultrasound image itself. The shown embodiment is described by taking the latter as an example, where the terminal device is an ultrasound imaging device.

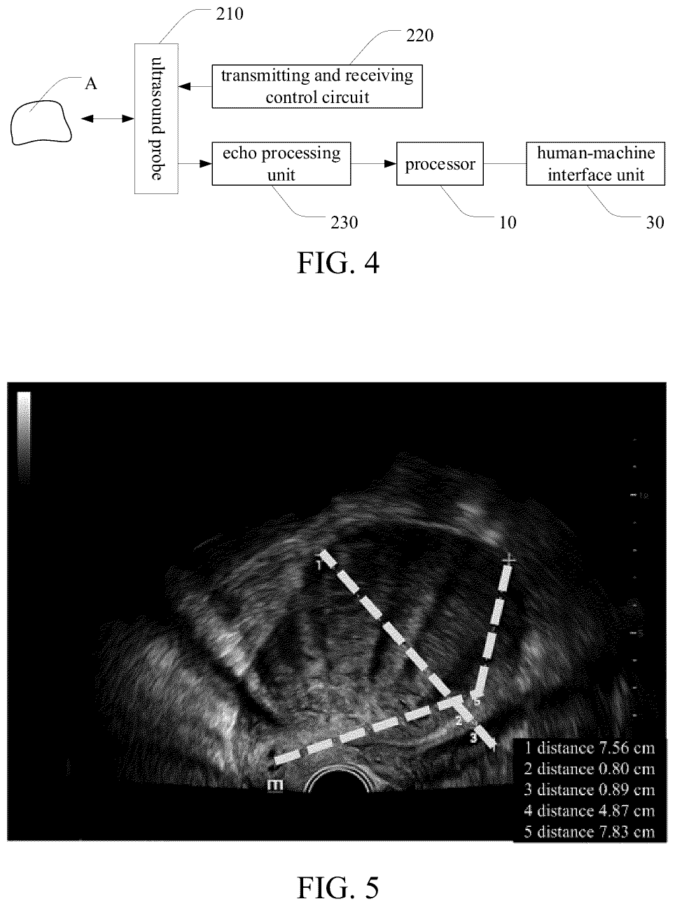

As shown in FIG. 4, the acquisition unit 20 of the ultrasound imaging device comprises an ultrasound probe 210, a transmitting and receiving circuit 220, and an echo processing unit 230.

The ultrasound probe 210 is configured to emit ultrasound waves toward a target tissue A (region of interest) and receive corresponding ultrasound echo signals to obtain ultrasound data, such as two-dimensional or three-dimensional ultrasound data. In some embodiments, the ultrasound probe 210 comprises multiple transducer elements configured to achieve mutual conversion between electrical pulse signals and ultrasound waves, thereby enabling emission of ultrasound waves toward the target tissue A and reception of corresponding ultrasound echo signals. Each transducer element may be configured to emit ultrasound waves based on excitation electrical signals or convert received ultrasound waves into electrical signals. Accordingly, each transducer element can be used to emit ultrasound waves towards the target tissue A or receive ultrasound echoes returned by the tissue. During ultrasound detection, transmission sequences and reception sequences may be employed to control which transducer elements are used for emitting ultrasound waves and which are used for receiving ultrasound waves, or to allocate transducer elements to specific time slots for emitting ultrasound waves or receiving ultrasound echoes. All transducer elements participating in ultrasound emission may be simultaneously excited by electrical signals to emit ultrasound waves concurrently, or may be excited by multiple electrical signals with specific time intervals to continuously emit ultrasound waves with defined temporal spacing.

The transmitting and receiving circuit 220 is configured to control the ultrasound probe 210 to perform both ultrasound wave emission and ultrasound echo signal reception. For example, the transmitting and receiving circuit 220 is configured to control the ultrasound probe 210 to emit ultrasound waves toward the target tissue A, and to control the ultrasound probe 210 to receive ultrasound echo signals reflected from the region of interest. In some embodiments, the transmitting and receiving circuit 220 is configured to generate transmission sequences and reception sequences, and output them to the ultrasound probe 210. The transmission sequences are configured to control some or all transducer elements among the multiple transducer elements in the ultrasound probe 210 to emit ultrasound waves toward the target tissue A. Parameters of the transmission sequences include the number of activated transducer elements for emission and ultrasound wave transmission parameters (e.g., amplitude, frequency domain, number of transmissions, transmission interval, emission angle, waveform, and/or focal position). The reception sequences are configured to control some or all transducer elements among the multiple transducer elements to receive echoes of ultrasound waves after tissue interaction. Parameters of the reception sequences include the number of activated transducer elements for reception and echo reception parameters (e.g., reception angle, imaging depth). Depending on the application of the ultrasound echoes or differences in images generated from the ultrasound echoes, the ultrasound wave parameters in the transmission sequences and the echo parameters in the reception sequences may correspondingly differ.

The echo processing unit 230 is configured to process ultrasound echo signals received by the ultrasound probe 210, such as filtering, amplifying, and beamforming on the ultrasound echo signals to generate ultrasound image data. In specific embodiments, the echo processing unit 230 may output the ultrasound image data to the processor 10 or first store the ultrasound image data in the memory 40. When operations need to be performed based on the ultrasound image data, the processor 10 reads the ultrasound image data from the memory. Those skilled in the art should appreciate that in some embodiments, the echo processing unit 230 may be omitted when processing such as filtering, amplification, or beamforming of the ultrasound echo signals is not required. In other embodiments, some or even all of the functions of the echo processing unit 230 may be implemented by the processor 10, that is, the echo processing unit 230 may form a part of the processor 10.

The processor 10 is configured to acquire ultrasound image data and apply relevant algorithms to obtain required parameters or images. In some embodiments of the present disclosure, the processor 10 comprises, but is not limited to, devices such as a central processing unit (CPU), a microcontroller unit (MCU), a field-programmable gate array (FPGA), and a digital signal processor (DSP), which are configured to interpret computer instructions and process data in computer software. In some embodiments, the processor 10 is configured to execute computer application programs stored in a computer-readable storage medium, thereby implementing various functions of the ultrasound imaging device. The ultrasound image of the target section in this embodiment refers to beamformed ultrasound image data. For example, it may be ultrasound image data that cannot be directly displayed on a display without corresponding processing, or ultrasound image data that can be directly displayed on the display after corresponding processing.

The HMI unit 30 is configured to facilitate human-computer interaction, including receiving user input and outputting visual information. It can receive user input via keyboards, operation buttons, mice, trackballs, or a touchscreen integrated with the display. Its output visual information may be displayed on a display. The display is configured to present information such as parameters and images calculated by the processor 10. Those skilled in the art should appreciate that in some embodiments, the ultrasound imaging device itself may not integrate a display but instead connect an external display device for information presentation.

It should be noted that the structure illustrated in FIG. 4 is only illustrative, and may include more or fewer components than those shown in FIG. 4, or may have configurations different from those shown in FIG. 4. The components illustrated in FIG. 4 may be implemented using hardware and/or software.

The ultrasound image of the target section may be a two-dimensional ultrasound image, which can be obtained by scanning with an ultrasound probe, or can be obtained by first acquiring three-dimensional ultrasound data and then slicing based on the three-dimensional ultrasound data. This embodiment uses a two-dimensional ultrasound B-mode image as an example for illustrative purposes. The echo processing unit 230 can process ultrasound echo signals to directly obtain the ultrasound image of the target section, that is, users can acquire the ultrasound image of the target section through routine B-mode scanning on the target tissue A. Alternatively or additionally, the echo processing unit 230 may process ultrasound echo signals to obtain a three-dimensional ultrasound image of the target tissue, and than the processor 10 can obtain a cross-sectional image of the target section or a rendered image from the three-dimensional ultrasound data, thereby obtaining the ultrasound image of the target section.

The at least one target section of the target tissue may include at least one of the sagittal plane, transverse plane, or coronal plane of the target tissue. This embodiment takes these three planes as an example for illustration, and subsequently generates lesion distribution diagrams corresponding to these three planes. Since these three planes are perpendicular to each other, doctors can intuitively and accurately grasp the spatial distribution of lesions in the target tissue by analyzing the lesion distribution diagrams across these three planes.

After obtaining the ultrasound image of the target section, the processor 10 can display the ultrasound image on the display of the HMI unit 30. As mentioned above, one implementation of step 11 involves using the ultrasound probe 210 to scan the target tissue with ultrasound to obtain the ultrasound image of the target section and display it. However, after detecting the lesion, doctors often need to observe and measure both the lesion and its surrounding target tissue in detail. Consequently, step 11 may correspond to a routine ultrasound scanning procedure performed by the ultrasound imaging system on the target tissue and its lesions, eliminating the need for additional ultrasound scans.

The target tissue may be various biological tissues, including but not limited to the uterus, heart, thyroid, ovaries, kidneys, or liver. This embodiment takes the uterus as an example for explanation. During the examination of a patient's uterine, the patient assumes the lithotomy position for routine transvaginal two-dimensional ultrasound examination to determine the position and basic situation of various anatomical structures of the uterus (such as endometrium, uterine body, and cervix). Measurements and observations are performed on the endometrium and uterine body in preserved sagittal and transverse uterine planes. The ultrasound image of the sagittal uterine is illustrated in FIG. 5. When observing the coronal plane of the uterus, transvaginal three-dimensional ultrasound scanning is conducted. Before scanning, the scanning angle and scanning position of the volumetric vaginal probe are adjusted to ensure complete coverage of all anatomical structures. After the scan is completed, the sagittal, transverse, and coronal uterine images are extracted from the stored three-dimensional dataset through manual sampling frame adjustment or automated detection algorithms for further measurement and analysis. Afterwards, a two-dimensional sector scanning or three-dimensional data reconstruction can be performed to observe the distribution of uterine fibroids. The above process can also be completed through transabdominal ultrasound examination.

Step 12: generating a target tissue diagram of the target section by the processor 10 based on the ultrasound image of the target section.

The target tissue diagram of the target section is a schematic diagram designed to depict the morphological feature (e.g., contour) of the target tissue on the target section. There are various methods for generating the target tissue diagram, several of which are exemplified below. The generated target tissue diagram is closer in morphology to the target tissue shown in the ultrasound images.

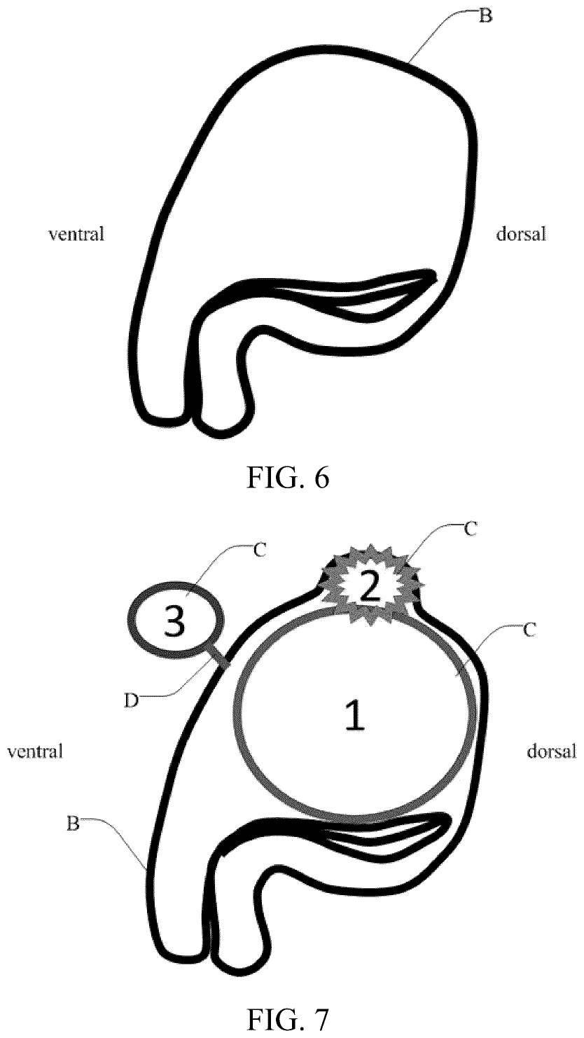

Method I: The processor 10 segments the contour of the anatomical structure of the target tissue from the ultrasound image of the target section, and generates the target tissue diagram of the target section based on the contour of the anatomical structure of the target tissue. FIG. 6 illustrates a target tissue diagram B of the uterus in the sagittal plane. In this embodiment, there are three target sections: the sagittal, transverse, and coronal planes, so the target tissue diagrams of these three sections are generated separately. There may be one or more anatomical structure of the target tissue, which also referred to as key anatomical structures. These structures represent primary regions of the target tissue that exhibit sufficient specificity for users to identify the type of target tissue, with a certain degree of recognition and representativeness. For example, the endometrium, cervix, and uterine body constitute the anatomical structure of the uterus. Observing at least one of these three structures allows users to know that the target tissue is the uterus. For another example, the anatomical structure of the heart include one or more of the following: left ventricle, left atrium, right ventricle, right atrium, superior vena cava, inferior vena cava, myocardial wall, valves, and aortic arch. The anatomical structure of the thyroid includes the left thyroid lobe and/or the right thyroid lobe. The anatomical structure of the ovary includes the outer contour of the ovary and/or follicles. The anatomical structure of the liver includes one or more of the left lobe, middle lobe, and right lobe.

For some target tissues, such as the uterus, scanning a single ultrasonic image may fail to obtain an image of the entire uterus. Therefore, multiple ultrasound images of the target section may thus be obtained (e.g. obtained through repeated scanning, or even scans acquired with different probes), and then the contour of the anatomical structure can be segmented from these multiple ultrasound images. Alternatively, for the anatomical structures with small morphological differences across individuals (e.g. the cervix), a template map may be used as a substitute. Taking the latter as an example, this type of target tissue may be labeled as a first-type target tissue, which is pre-associated with a template map of its first anatomical structure. The type of target tissue (i.e., what type of tissue the target tissue is) is predefined, for example, the processor 10 may recognize the type of target tissue by analyzing the ultrasound image of the target tissue, or may determine the type of target tissue based on user input, or may determine the type of target tissue based on a pre-selected examination mode by a user, and then check whether the type of target tissue is the first-type target tissue. If so, the contour of the anatomical structure of the target tissue is segmented from the ultrasound image of the target section, and the template map of the first anatomical structure associated with the first-type target tissue is acquired in advance. The segmented contour of the anatomical structure and the obtained template map of the first anatomical structure are integrated to generate the target tissue diagram of the target section. Taking the uterine scenario as an example, the anatomical structure that need to be segmented includes the endometrium and uterine body, which are then combined with a predetermined cervical template map to automatically generate a personalized target section schematic diagram (target tissue diagram). Here, “personalized” means that the morphology of the target tissue diagram matches the patient's actual anatomy (target tissue), and variations exist across different patients' target tissue diagrams.

The processor 10 can implement the aforementioned structural segmentation through various methods. For example, detection of the endometrium and uterine body can be achieved based on traditional feature detection methods such as grayscale and/or morphological characteristics. Machine learning or deep learning methods may also be employed to detect or precisely segment the corresponding anatomical structures in the ultrasound images. Several segmentation approaches are described below as examples.

One approach involves detecting the anatomical structure based on conventional methods. In the aforementioned ultrasound image, the endometrium and uterine body exhibit distinct echogenic difference boundaries from surrounding tissues, enabling contour detection of the endometrium and uterine body through traditional morphological and other feature detection methods. For instance, the processor 10 first performs binary segmentation on the ultrasound image, performs a predetermined morphological operation to obtain multiple candidate regions, and then determines the probability of containing a anatomical structure in each candidate region based on its shape, grayscale intensity, and other features. The region with the highest probability is identified as containing the anatomical structure. Within this region, morphological features are subsequently utilized to detect the contour of the target anatomical structure, thereby obtaining its outline. Of course, besides morphological feature detection, other conventional grayscale detection and segmentation methods may also be employed, including but not limited to OTSU's threshold method, Level Set, Graph Cut, and Snake algorithms.

Another approach involves detecting the anatomical structure through machine learning or deep learning methods. This method primarily applies machine learning or deep learning algorithms to the aforementioned ultrasound images, enabling algorithms to autonomously detect or precisely segment the anatomical structure by learning to construct models through data patterns. The method may comprise: establishing an expert database, training algorithms to learn the features or patterns of target regions in the database, and automatically detecting and segmenting of the anatomical structure on new data based on the learned knowledge. Specifically, in the present disclosure, the method may include the following steps:

### 1) Establishing an Expert Database

The expert database refers to a collection of ultrasound images of multiple target tissue sections in which anatomical structures have been manually delineated as target regions based on the knowledge and experience of experts. These annotated ultrasound images form the database for training machine learning or deep learning models. For example, in the context of endometrial detection and segmentation, the present disclosure involves manual annotation of endometrial regions as target regions first.

### 2) Key Anatomical Structure Detection and Segmentation

The ultrasound images from the expert database are input into a predetermined machine learning or deep learning model for training, resulting in a trained machine learning or deep learning model. Specifically, based on the existing expert database calibration, machine learning and/or deep learning algorithms (models) are applied to learn the ultrasound images in the expert database, to obtain the features or patterns that distinguish the anatomical structure (e.g., endometrium) from other regions, thereby enabling automated detection and segmentation of the anatomical structure. The specific implementation of automatic detection and segmentation for the anatomical structure includes but is not limited to the following methods:

The first method may employ traditional feature extraction combined with classifier discrimination. For example, sliding window-based approaches commonly used in conventional methods: the model first performs feature extraction on regions within the sliding window, where feature extraction methods could include traditional PCA, LDA, Haar features, texture features, or deep neural networks. The extracted features are then matched with an expert database and classified using discriminators such as KNN, SVM, random forest, or neural networks to determine whether the current sliding window represents the region of interest (the anatomical structure) while obtaining its corresponding classification.

The second method may utilize deep learning-based Bounding-Box detection and recognition, typically implemented as: the model learns features and regresses parameters through stacked convolutional layers and fully connected layers using constructed expert databases. For an input ultrasound image, the Bounding-Box of the corresponding region of interest (anatomical structure) can be directly regressed through the network, and the category of tissue structure within the region of interest can be obtained. Common networks include R-CNN, Fast R-CNN, Faster R-CNN, SSD, YOLO, etc.

The third method involves an end-to-end semantic segmentation network based on deep learning, which is similar in structure to the second deep learning-based Bounding-Box method. The difference is that it removes the fully connected layers and incorporating upsampling or deconvolution layers to make the input and output dimensions the same, thereby directly obtaining the region of interest (anatomical structure) and its corresponding category of the input image. Common networks include FCN, U-Net, Mask R-CNN, etc.

The fourth method is to employ only the first, second, or third method to locate a target, and then design an additional classifier based on localization results to classify and judge the target. Typical classification approaches include: first performing feature extraction on target ROI or Mask regions using traditional methods (PCA, LDA, Haar features, texture features) or deep neural networks, then matching extracted features with databases, and finally classifying using discriminators such as KNN, SVM, random forest, or neural networks.

The processor 10 inputs the ultrasound image of the target section into the trained machine learning or deep learning model, which outputs the contour of the anatomical structure. By using machine learning or deep learning algorithms to accurately segment the anatomical structure such as the endometrium, the accuracy of contour of each anatomical structure in the target tissue diagram is enhanced.

Method II: By marking several key points on an ultrasound image, corresponding target tissue diagrams (structural schematic diagrams) are automatically generated based on the key points and a predetermined basic tissue structure diagram. The method for automatically generating corresponding target tissue diagrams based on key points and the predetermined basic tissue structure diagram may employ approaches such as template matching. The relative positional relationships between key points obtained from the ultrasound image are mapped onto the predetermined basic tissue structure diagram, obtaining a personalized uterine structural schematic diagram (target tissue diagrams) through mapping transformations.

Specifically, the processor 10 displays multiple predetermined basic tissue structure diagrams of the target section on the display interface for user selection. These basic tissue structure diagrams can be understood as templates for target tissue diagrams. The ultrasound image of the target section is also displayed on the display interface, allowing users to input a plurality of key points (e.g., 7 points) on the ultrasound image. These key points are used to identify the position and dimension of the anatomical structure of the target tissue diagram, allowing users to outline the contour of the anatomical structure by simply marking key points according to the ultrasound image. Users may also place key points before modifying or deleting them. The processor 10 can map the positional relationships between the points input by a user to a user-selected basic tissue structure diagram, obtaining the corresponding contour of the anatomical structure, and subsequently generate the target tissue diagram of the target section based on these contours. The target tissue diagram obtained in this way can effectively reflect the uterine contour in corresponding sections of the patient.

The quantity of marked key points can be customized. In this embodiment, they are used to indicate the contour of endometrial and uterine body. The predetermined basic tissue structure diagram is used to represent various fundamental morphological presentations of target tissues in sagittal, transverse, and coronal planes. Taking the uterus as an example, it includes but is not limited to tissue structure diagrams corresponding to anteverted, retroverted, unicornuate, and bicornuate morphologies. Multiple basic tissue structure diagrams illustrate various types of uterine sections in sagittal, transverse, and coronal planes. These diagrams may be rendered using black lines during drawing the basic tissue structure, or alternatively implemented with other colors, line styles, or enhanced internal textures.

Method III: The target tissue diagram is generated automatically through measurements for key anatomical structures. The processor 10 can display multiple predetermined basic tissue structure diagrams of the target section on the display interface for user selection. It can acquire the dimensional measurement of the anatomical structure of the target tissue from the ultrasound image of the target section. FIG. 5 is the ultrasound image where the anatomical structure is measured, and the dashed lines in the figure correspond to dimensional measurement. The dimensional measurement of the anatomical structure are then mapped to the user-selected basic tissue structure diagram to generate the target tissue diagram of the target section.

Taking the distribution of uterine fibroid as an example, the dimensional measurement of the anatomical structure includes endometrial thickness, anterior posterior diameter of the uterine body, longitudinal diameter of the uterine body, and cervical length. Based on such dimensional measurement combined with the user-selected basic tissue structure diagram, a personalized structural schematic diagram (the target tissue diagram) is automatically generated through mapping or stretching transformation. The dimensional measurement is typically obtained by measuring on the ultrasound image or by user input. If the ultrasound image is unavailable, making it impossible to measure the dimensional measurement of the anatomical structure, a user-input value can serve as the dimensional measurement.

In other embodiments, doctors may directly select a predetermined basic tissue structure diagram as the target tissue diagram. For example, the processor 10 can display multiple predetermined basic tissue structure diagrams of the target section on the display interface for user selection, with the chosen basic tissue structure diagram serving as the target tissue diagram of the target section. However, the target tissue diagram generated in this way fails to reflect structural and dimensional variations in the same target tissue across different patients.

The generated target tissue diagrams from the aforementioned methods may be rendered using lines-such as black solid lines—or implemented through other colors, line styles, or enhanced internal textures. Various rendering methods are permissible, as the present disclosure is not limited to them.

From the various methods mentioned above, it can be seen that the contour of the generated target tissue diagram matches the target tissue in the ultrasound image. In order to reflect actual dimension of the target tissue, the ratio between the target tissue diagram of the target section and the ultrasound image of the target section may be set to 1:1 for easy comparison with the ultrasound image. Of course, other ratios may be adopted in other embodiments.

Step 2: taking the target tissue diagram of the target section as the background of the lesion distribution diagram of the target section, and displaying the target tissue diagram on the display interface.

For example, the processor 10 displays the target tissue diagram B of the target section as the background of the lesion distribution diagram of the target section on the display interface of the display, so as to provide reference for users to edit the lesion diagram in the lesion distribution diagram. With the target tissue diagram serving as the background, users can conveniently set parameters such as the position and dimension of the lesion diagram.

Step 3: determining a morphological parameter of the lesion diagram based on a user editing operation.

The morphological parameter includes at least one of appearance, dimension, orientation, and relative position to the target tissue diagram. The lesion diagram is a schematic diagram of the lesion. For example, the processor 10 can receive user editing operations through the HMI unit to determine the morphological parameter of the lesion diagram. The target tissue may have one or more (two or more) lesions, typically requiring the same number of lesion diagrams as lesions. The morphological parameter of the lesion diagram may be fully user-edited, partially user-edited (e.g., some parameters automatically generated while others are user-edited), or partially/fully auto-generated with subsequent user editing (adjustment) of at least a subset of parameters.

Step 4: based on the morphological parameter of the lesion diagram, displaying the lesion diagram in the corresponding region of the target tissue diagram by the processor 10, and obtaining the lesion distribution diagram of the target section, as illustrated in FIG. 7.

In this embodiment, the ultrasound image of the target section is two-dimensional, and the corresponding lesion distribution diagram is also a two-dimensional image. The morphological parameter includes at least the relative position between the lesion diagram and the target tissue diagram, which may be selected by users or preset by the system, where the “corresponding region” refers to this relative position. If the lesion is outside the target tissue, the lesion diagram is rendered outside the target tissue diagram; if a lesion is within the target tissue, the lesion diagram is rendered inside the target tissue diagram, maintaining a one-to-one correspondence therebetween. In Step 2, only the target tissue diagram of a single target section may be displayed at a time. Subsequently, in steps 3 and 4, the lesion distribution diagram of that specific target section is displayed. By repeating steps 1-4, the lesion distribution diagram for the next target section is obtained. That is, by scanning one target section, the lesion distribution diagram of that target section can be obtained through steps 1-4, with separate scans performed for different target sections to generate their respective lesion distribution diagrams. Of course, in step 2, all target tissue diagrams of the target section can also be displayed, allowing users to edit lesion diagrams on any desired target tissue diagrams. In summary, in this embodiment, the lesion distribution diagrams in the sagittal, transverse, and coronary planes can be obtained, which is equivalent to obtaining three views of the target tissue and the lesion, thereby providing a clear and comprehensive representation of lesion distribution. The workflow shown in FIG. 2 can be completed under the operation of a sonographer. Specifically, when a sonographer examines a tissue/organ and its lesion, based on the process shown in FIG. 2, only editing operations are needed to generate a lesion distribution diagram that reflects pathological spatial relationships, which can then be provided to clinicians to precisely communicate lesion distribution characteristics.

The lesion diagram, as the schematic diagram of the lesion, may specifically be configured to illustrate the morphology of the lesion on the target section, which is equivalent to outlining both the contours of the target tissue and lesion in the ultrasound image of the target section. Since the lesion may locate at any position in the target tissue rather than aligning with its geometric center, the morphology of the lesion on the target section often fails to reflect its true length, width, or height, resulting in inaccurate dimensional representation. Accordingly, further improvement can be made. In this embodiment, the lesion diagram can be a schematic diagram that can reflect the dimension of the lesion. Here takes a uterine in the sagittal plane as an example. In the lesion distribution diagram of the uterine in sagittal plane, its target tissue diagram represents the uterine in the sagittal plane, while the lesion diagram reflects the length and width of the lesion. In the coronal and transverse planes, the lesion diagram in the lesion distribution diagram reflects length-height and height-width dimensions. In this way, regardless of the spatial position of the lesion within the target tissue, the lesion distribution diagrams in the sagittal, transverse, and coronal planes can collectively convey the proportional size of the lesion relative to the target tissue and the spatial positioning of the lesion within the target tissue. It can be seen that the lesion distribution diagrams can efficiently synthesize both dimensional and positional data, enabling clinicians to rapidly assimilate critical diagnostic information about lesion characteristics and spatial relationships.

There are various ways for users to determine the morphological parameters of the lesion diagrams through multiple approaches, as exemplified below.

Users can manually select the appearance of the lesion diagram and position it. For example, editing operations may include an appearance selection operation and a region selection operation. A display interface showing a target tissue diagram displays multiple appearance identifiers of different appearances for user selection, where the appearances represent a lesion type being benign or malignant, and a lesion contour. The appearance identifiers could be text descriptions or lesion diagrams reflecting the appearances, provided they uniquely identify the appearances. This embodiment takes lesion diagrams as appearance identifiers for illustration. As shown on the left side of FIG. 8, a plurality of lesion diagrams with different appearances are provided for user selection, where the lesion diagrams C1-C3 are different in appearance. The appearances of the lesion diagrams may include two attributes: contour (representing lesion outline) and/or whether the contour is smooth (indicating benign or malignant). As shown in the figure, the appearance of the lesion diagram C1 exhibits a smooth elliptical appearance with two elements: elliptical shape (indicating ovoid morphology of the lesion) and smooth elliptical contour (denoting benign lesion); the appearance of the lesion diagram C2 shows a non-smooth elliptical appearance with two elements: elliptical shape (indicating ovoid morphology) and irregular contour (denoting malignant lesion). For lesion diagrams indicating malignancy, their appearance may further incorporate a malignancy level as a sub-element. In other words, in addition to distinguishing between benign and malignant classifications, some embodiments may further classify the severity of malignancy (e.g., borderline, malignant grade 1, malignant grade 2). For example, the displayed plurality of lesion diagrams with different appearances include: lesion diagrams with benign-indicating appearances, and multiple lesion diagrams with appearances respectively characterizing various malignancy levels. When a lesion is non-benign, users may directly select a lesion diagram corresponding to its malignancy level. In some embodiments, lesion diagrams may include a third element: lesion stalk. The line segments at the lower-right corners of lesion diagrams C3 and C4 represent lesion stalks, which means that users may determine the appearance of the lesion diagram by selecting at least one parameter from: benign/malignant status, contour features, and presence/absence of a lesion stalk.

Users may select appropriate lesion diagrams based on ultrasound scanning of the lesion performed in advance, thereby determining the appearance within the morphological parameters. The processor 10, in response to an appearance selection operation, takes the selected lesion diagram as the lesion diagram in the lesion distribution diagram. In some embodiments, the display interface may not present multiple appearance identifiers for user selection but instead allows direct user input of the appearance parameter to define the appearance of the lesion diagram.

Both external and internal regions of the displayed target tissue diagram are selectable by users. Based on a region selection operation, the user-selected region-defining the relative position between the lesion diagram and the target tissue diagram—is determined. The processor 10 displays the lesion diagram in the user-selected region. For instance, users may move the cursor to a desired location (region) on the target tissue diagram and click to select the region. In this embodiment, a user can directly place (or drag-and-drop) a lesion diagram onto the target tissue diagram to generate the lesion distribution diagram. As shown in FIG. 8, after selecting a lesion diagram of specific appearance, the user drags it to a region of the target tissue diagram, providing an intuitive and convenient method for positioning the lesion diagram. In some embodiments, the processor 10 may automatically obtain the relative position between the lesion and target tissue from ultrasound images of the target tissue and/or lesion, establishing this as the initial relative position between the lesion diagram and the target tissue diagram. Editing functionality is then provided to allow users to modify this initial position to create a new relative position. Users may retain the initial position without further editing if deemed appropriate.

The size of the lesion diagram, primarily its scale relative to the target tissue diagram, may be preset for subsequent user editing to match the lesion dimensions or automatically determined, with the latter being exemplified in this embodiment. During the ultrasound scanning of the lesion described in earlier steps, ultrasound images of the lesion are acquired. Typically, the maximum dimensions of the lesion in three orthogonal directions (XYZ: length, width, height) may be measured by users to obtain dimensional measurements, which may include one or more of the following: lesion length, width, and height. As the lesion diagram in this embodiment is two-dimensional, only two of these measurements are required. This allows the same lesion diagram across two lesion distribution diagrams to collectively reflect the lesion's three-dimensional size, with the dimensional measurements representing its actual dimensions. The processor 10 retrieves the dimensional measurements of the lesion from its ultrasound images of the lesion and further determines the dimensions of the lesion diagram according to the dimensional measurements. The relationship between the dimensional measurements and the dimensions of the lesion diagram are typically proportional, such as 1:1 (actual size ratio), <1:1 (the lesion diagram reduced relative to the lesion, indicating an enlarged lesion diagram), or >1:1 (the lesion diagram enlarged relative to the lesion, indicating a reduced lesion diagram). In the lesion distribution diagram, the target tissue diagram and lesion diagram may maintain a consistent scale ratio. For example, the target tissue is represented at a 1:2 scale (actual tissue to diagram), and the lesion diagram is also represented at a 1:2 scale (actual lesion to diagram). This creates a 1:1 proportional relationship between the target tissue diagram and lesion diagram. Consequently, the schematic directly visualizes the actual size of uterine fibroid relative to the uterus, providing an intuitive representation.

The orientation of the lesion diagram may be defined by the direction of the ellipse's major axis (i.e., its lengthwise direction). A preset initial orientation, such as horizontal orientation, may be applied. This orientation may be subsequently edited by users if adjustment is required.

The morphological parameters mentioned above, including appearance, dimensions, orientation, and relative positioning, may be automatically obtained or preset for parameters not manually edited by the user. The methods for determining these parameters (user editing, automated acquisition, and preset configurations) have been exemplified in preceding sections. The processor 10 subsequently renders the lesion diagram based on the resulted morphological parameters.

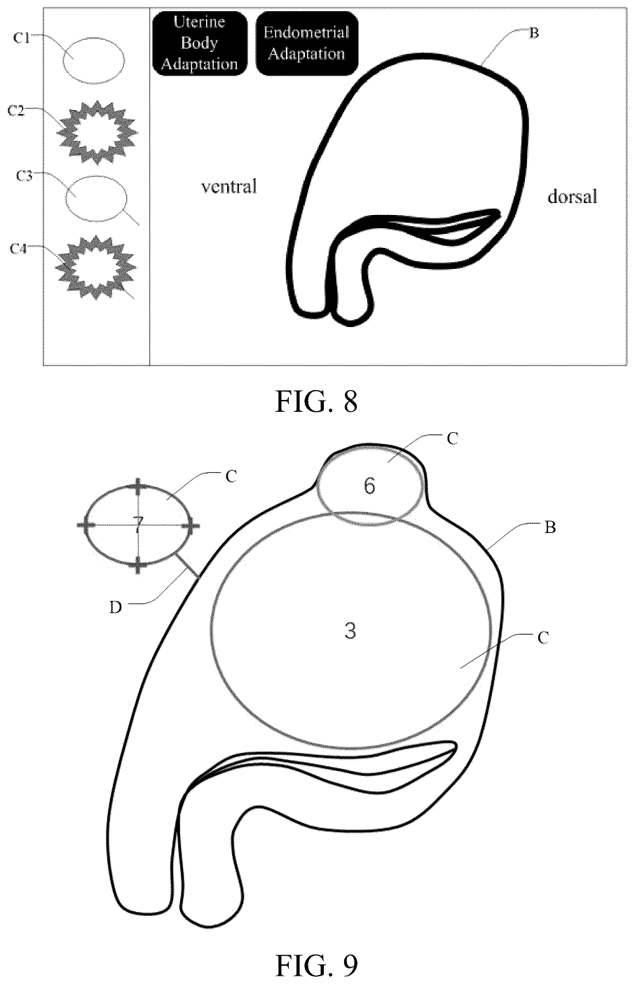

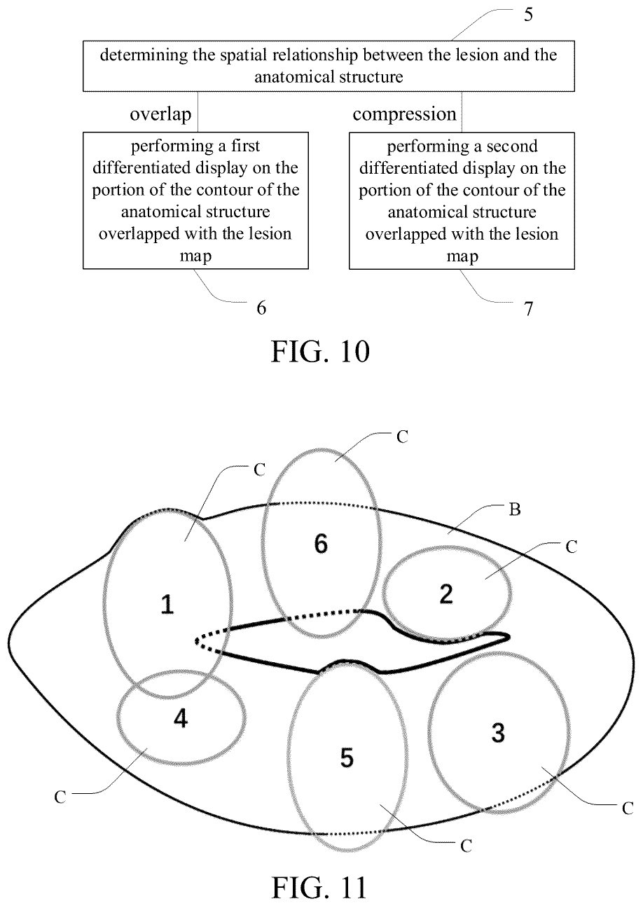

For the displayed lesion diagram in the lesion distribution diagram, operations such as deletion and editing can be performed subsequently by users, facilitating modification of lesion diagrams and making the drawing of the lesion distribution diagram highly convenient. Correspondingly, the processor 10 deletes the displayed lesion diagram based on a user-initiated operation for deleting a selected lesion diagram. The processor 10 adjusts morphological parameter of the lesion diagram based on a user editing operation and updates the displayed lesion diagram according to the adjusted morphological parameters. The processor 10 modifies the morphological parameter of the lesion diagram through a user editing operation. For example, the lesion diagram in the lesion distribution diagram can be selected by users; and the editing operation can comprise a moving operation, where a user can move a selected lesion diagram to any position in the lesion distribution diagram through the HMI unit. The processor 10 correspondingly adjusts the relative position between the lesion diagram and the target tissue diagram by executing these movement operations on the displayed lesion diagram by a user (the selected lesion diagram within the lesion distribution diagram). Such movement operations by users may involve cursor selection and dragging of lesion diagrams. Editing operations may further include stretching or scaling functions. The processor 10 stretches or scales the displayed lesion diagrams according to user stretching or scaling operations respectively, thereby modifying their appearance and/or dimensions. Rotation operations are also supported, with the processor 10 rotating displayed lesion diagrams based on rotation instructions by users to adjust the orientation of respective lesion diagram. As shown in FIG. 9, when a lesion diagram in the lesion distribution diagram is selected (a lesion diagram 7 selected in the figure), multiple edit points (i.e. cross symbols in FIG. 9) become visible for user modification. Adjusting these edit points enables modification of the appearance, size, and/or orientation of the lesion diagram. For example, when the cursor clicks on the leftmost edit point, this edit point follows the cursor's positional changes. The size and orientation of the elliptical contour representing lesion diagram C are correspondingly altered, equivalent to performing rotation and stretching operations with the rightmost edit point as the coordinate origin. This method provides efficient size and orientation adjustments of the lesion diagram. These editing operations may also be applied during step 3, though detailed description is omitted here.

The lesion and the anatomical structure of the target tissue may spatially overlap or compress each other. These spatial relationships are visually represented in the lesion distribution diagram, enabling clinicians to intuitively observe the interactions through imaging. The specific process, as illustrated in FIG. 10, includes the following steps:

Step 5: determining the spatial relationship between the lesion and the anatomical structure.

Specifically, the target tissue diagram B of the target section includes the contour of the anatomical structure of the target tissue. The processor 10 receives user-defined spatial relationships between the lesion and these anatomical structures. If multiple anatomical structures are present near the lesion, the spatial relationships between the lesion and each structure are required to be specified. In this embodiment, the spatial relationships are categorized as overlap or compression. The display interface may also display the anatomical structure of the target tissue and possible spatial relationships for user selection. The selected anatomical structure and the selected relationship are then linked to define the spatial relationship between the lesion and the selected anatomical structure. As shown in FIG. 8, when a “Uterine Body Adaptation” virtual button is activated, the spatial relationship between the lesion and the uterine body is set as compression; and if the button is inactive, the spatial relationship between the lesion and the uterine body is set as overlap, where placing the lesion diagram directly on the target tissue diagram represents overlap. Accordingly, overlap is regarded as a default spatial relationship, streamlining user input for common overlap scenarios. Similarly, selecting the “Endometrial Adaptation” button defines the lesion's relationship with the endometrium (another anatomical structure) as compression, while deselection reverts to overlap.

Step 6: when the spatial relationship exhibits overlap, performing a first differentiated display on the portion of the contour of the anatomical structure overlapped with the lesion diagram by the processor 10 to visually represent this overlapping spatial relationship between the lesion and the anatomical structure.

Step 7: when the spatial relationship exhibits compression, performing a second differentiated display (e.g., ensuring no overlap) on the portion of the contour of the anatomical structure overlapped with the lesion diagram by the processor 10, thereby indicating the compressive spatial relationship. Notably, the first and second differentiated displays are different.

Steps 5-7 can be performed at any time, such as before Step 3 or 4. When the user places the lesion diagram or moves the lesion diagram, the contour of the target tissue diagram at the location of the lesion diagram may be adaptively blurred or adjusted according to the spatial relationship, thereby presenting the spatial relationship between them. If performed after Step 4, the target tissue diagram B may change from the contour shown in FIG. 8 to the contour shown in FIG. 9.

It can be seen that after the user sets the spatial relationship, the present embodiment can adaptively adjust the contours of anatomical structures in the target tissue diagram to present the spatial relationship between the lesion and said anatomical structures based on different placement positions of the lesion diagram, which is both convenient and intuitive.

Some tumors have stalks, through which the tumors attach to tissues. These stalks can also be represented in the lesion distribution diagram. Specifically, in addition to the target tissue diagram and the lesion diagram, the lesion distribution diagram may include a lesion stalk diagram. The lesion stalk diagram can be generated automatically or manually, as described below.

The processor 10 can generate the lesion stalk diagram D and display the diagram D between the lesion diagram C and the target tissue diagram B based on their relative positions, as shown in FIGS. 7 and 9. The lesion stalk diagram D connects the lesion diagram C at one end and the target tissue diagram B at the other. Specifically, when the lesion diagram C is located outside the target tissue diagram B (i.e., the lesion is outside the target tissue), the processor 10 generates the lesion stalk diagram D and displays it between the lesion diagram C and the target tissue diagram B. When the lesion diagram C is located within a cavity of the target tissue diagram B (i.e., the lesion is inside a cavity of the target tissue), the processor 10 similarly generates the lesion stalk diagram D and displays it between the lesion diagram C and the target tissue diagram B. No lesion stalk diagram is generated for other relative positions. Automatic generation of the lesion stalk diagram is efficient and user-friendly.

For manual creation, multiple approaches exist. For example, when selecting the appearance of the lesion diagram, a pre-designed appearance that includes the lesion stalk diagram may be directly chosen. For another example, the lesion stalk diagram may be drawn directly on the lesion distribution diagram by users. The processor 10 detects a user-initiated operation for drawing a lesion stalk diagram between the lesion diagram C and the target tissue diagram B, generates the lesion stalk diagram D in response, and displays it between the lesion diagram and the target tissue diagram. The lesion stalk diagram D can be represented by simple lines, making manual creation quick and straightforward.

For both automatically and manually generated lesion stalk diagram, the connection point at the other end attached to the target tissue diagram C may be: the closest point on the contour of the anatomical structure adjacent to the lesion diagram C. For example, the lesion stalk diagram may be represented as the shortest line segment between the lesion diagram C and the target tissue diagram B. The position of this connection point can be adjusted on the lesion distribution diagram. For instance, the processor 10 receives a user-initiated operation for selecting the connection point and moving it, then updates the connection point location to the moved position based on this operation.

In the prior art, to visualize the distribution of myomas relative to the endometrium and uterine wall, doctors may manually draw schematic diagrams of the uterine in the sagittal, transverse, and coronal planes and sketch lesions on them. This approach provides an intuitive representation of lesion distribution, facilitates clinical understanding, and effectively translates findings from ultrasound or other imaging modalities into clinically familiar visualizations. It is particularly helpful for determining appropriate surgical plans in cases such as uterine myomas, especially multiple myomas. However, this method relies on manual drawing by doctors. Since uterine morphology, size, and lesion characteristics vary across patients, manual diagrams cannot establish a one-to-one correspondence with imaging findings (e.g., ultrasound) to reflect individual variations. More critically, manual drawing is time-consuming and negatively impacts workflow efficiency. In contrast, with the device and method provided by the present disclosure, users can create lesion distribution diagrams quickly and conveniently while precisely reflecting individual anatomical variations in the patient's tissues and organs, significantly enhancing clinical workflow efficiency.

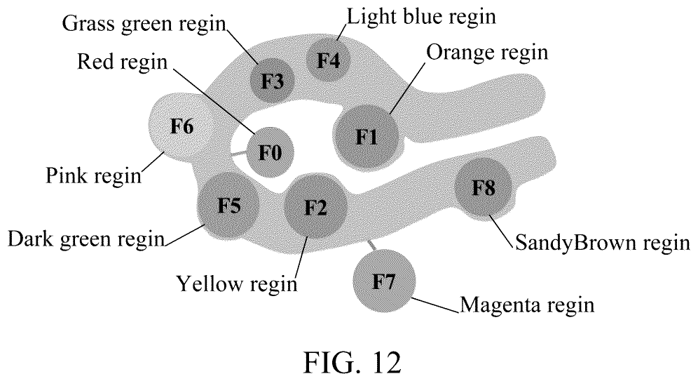

As shown in FIG. 12, the display interface of the display may also show a tumor classification guide diagram to prompt users to classify tumors. The tumor classification guide diagram includes schematic graphics of multiple tumor types (e.g., F0-F8 in the figure), each with distinctive image characteristics that uniquely identify the tumor type. In the figure, these image characteristics include classification labels (F0-F8) and colors, both of which unambiguously represent tumor types. In some embodiments, these image features may be represented by the colors labeled as “Red region”. “Orange region”, “Yellow region”. “Grass green region”, “Light blue region”. “Dark green region”, “Pink region”, “Magenta region”. “SandyBrown region” as shown in FIG. 12. For uterine applications, the tumor classification guide diagram may follow the International Federation of Gynecology and Obstetrics (FIGO) classification system, categorizing myomas into nine types (F0-F8 corresponding to FIGO Type 0 to Type 8). Each tumor schematic also depicts the relative positional relationships between the myoma and anatomical structures such as the endometrium and uterine wall.

Users can reference the tumor classification guide diagram to classify lesions, such as by inputting or selecting a tumor type. Each tumor type is pre-associated with specific image characteristics. The processor 10 determines the tumor type of the lesion based on the user's classification operation and displays the corresponding image characteristics (e.g., the assigned classification label and/or color) on the lesion diagram C.

The FIGO classifies myomas based on their relative positional relationships with the endometrium and uterine wall. Therefore, the processor 10 can determine the tumor type of the lesion according to the relative position between the lesion diagram and the target tissue diagram, following a preset classification rule, and then display the corresponding image characteristics of the determined tumor type on the lesion diagram. This provides a clear indication of the tumor type of the lesion.

Considering that the same lesion diagram may vary in shape and size across different lesion distribution diagrams, lesion diagrams can be assigned unique identifiers. For example, during the lesion diagram placement step, the processor 10 may automatically number the lesion diagrams based on their placement sequence (e.g., labeling the first placed myoma as “1” or “M1,” the second as “2” or “M2,” etc.). After completing a lesion distribution diagram of the target section, when creating lesion diagrams for subsequent lesion distribution diagrams of the target section, the processor 10 determines the identifier for each lesion diagram based on the positional relationship between the target sections and the relative location of the current lesion diagram. Users may also manually modify the identifiers using the HMI unit such as knobs, keyboards, or function keys. Since the same lesion may appear significantly different across schematic diagrams of different target sections, assigning identifiers to each lesion diagram allows easy identification of the same lesion across multiple schematic diagrams, significantly enhancing convenience.

In some embodiments, the processor 10 may further display a patient orientation corresponding to the lesion distribution diagram of the target section on the display interface based on the type of the target section. The displayed type of the target section is one of a sagittal plane, transverse plane, or coronal plane, and the displayed patient orientation includes at least one of left, right, ventral (anterior), dorsal (posterior), cranial (superior), or caudal (inferior). Specifically, the types of target sections include sagittal, transverse, and coronal planes. Patient orientations are divided into six directions: left, right, ventral, dorsal, cranial, and caudal, with each pair being opposing. The patient orientation effectively indicates the relative positional relationship between the target tissue map and the patient's body. As shown in FIGS. 6-8, for a sagittal plane, the corresponding patient orientations include ventral and dorsal, which characterize the relative positional relationship between the patient's abdomen/back and the target tissue diagram; for a coronal plane, the corresponding patient orientations may include cranial and caudal; and for a transverse plane, the corresponding patient orientations may include left and right. Additional patient orientations may also be displayed as needed.

This disclosure describes, with reference to various exemplary embodiments. However, Those skilled in the art will recognize that modifications and changes may be made to the exemplary embodiments without departing from the scope of this document. For instance, various operational steps, as well as the components used to execute these steps, may be implemented in different manners, taking into account specific applications or various cost functions related to system operation (e.g., one or more steps may be omitted, modified, or combined with other steps).

Additionally, as understood by those skilled in the art, the principles presented herein may be embodied in a computer program product reflected on a computer-readable storage medium, wherein the readable storage medium is pre-installed with computer-readable program code. Any tangible, non-transitory computer-readable storage medium may be utilized, including magnetic storage devices (hard disks, floppy disks, etc.), optical storage devices (CD-ROMs, DVDs, Blu-ray discs, etc.), flash memory, and/or the like. These computer program instructions can be loaded onto general-purpose computers, special-purpose comp uters, or other programmable data processing devices to form a machine, enabling the instructions executed on these computers or other programmable data processing devices to generate devices that perform specified functions. These computer program instructions can also be stored in a computer-readable memory, which can instruct a computer or other programmable data processing device to operate in a specific manner, such that the instructions stored in the computer-readable memory can form an article of manufacture, including an implementation device that realizes the specified functions. Furthermore, computer program instructions can be loaded onto a computer or other programmable data processing device, thereby executing a series of operational steps on the computer or other programmable device to produce a computer-implemented process. This allows the instructions executed on the computer or other programmable device to provide steps for realizing specified functions.

While the principles disclosed herein have been illustrated through various embodiments, it should be understood that structural configurations, material selections, and component proportions particularly suited to specific operational environments may be modified without departing from the scope and spirit of the disclosure. Such modifications, along with other adaptations or adjustments, shall be encompassed within the scope of the present disclosure.

The foregoing detailed description has been described with reference to various embodiments. However, those skilled in the art will recognize that modifications and variations may be made without departing from the scope of the disclosure. Accordingly, the description of the disclosure shall be interpreted in an illustrative rather than restrictive sense, and all such modifications are intended to be included within its scope. Similarly, discussions of advantages, alternative solutions to problems, and operational benefits associated with the embodiments are provided above. Nevertheless, benefits, advantages, solutions to problems, and any elements that may produce such effects or render them more explicit shall not be construed as critical, required, or essential. Furthermore, the term ‘coupled’ and its derivatives encompass physical connections (e.g., mechanical joints), electrical connections (e.g., circuit interconnects), magnetic linkages (e.g., inductive coupling), optical interfaces (e.g., fiber-optic alignment), communication channels (e.g., wireless protocols), functional integrations (e.g., software APIs), and any other form of association that achieves operational interaction.

Those skilled in the art will recognize that numerous modifications to the details of the above-described embodiments may be made without departing from the fundamental principles of the disclosed subject matter. Accordingly, the scope of the present disclosure shall be determined solely by the claims and their legal equivalents.

What is claimed is:

1. A method for presenting lesion distribution, comprising:
acquiring a target tissue diagram of at least one target section of a target tissue, wherein the target tissue diagram of the target section is a schematic diagram for presenting a morphology of the target tissue having at least one lesion on the target section;
displaying the target tissue diagram of the target section on a display interface;
determining a morphological parameter of a lesion diagram of a lesion in response to an editing operation, the morphological parameter comprising at least one of an appearance of the lesion diagram, a dimension of the lesion diagram, an orientation of the lesion diagram, and a relative position between the lesion diagram and the target tissue diagram; and
displaying the lesion diagram on a corresponding region of the target tissue diagram according to the morphological parameter of the lesion diagram, thereby obtaining the lesion distribution diagram of the target section.

2. The method of claim 1, wherein acquiring the target tissue diagram of at least one target section of the target tissue comprises:
obtaining an ultrasound image of the at least one target section of the target tissue; and
generating the target tissue diagram of the target section based on the ultrasound image of the target section.

3. The method of claim 1, wherein, the target tissue diagram of the target section comprises a contour of an anatomical structure of the target tissue, and the method further comprises:
receiving a spatial relationship between the lesion and the anatomical structure determined by a user;
when the spatial relationship exhibits overlap, performing a first differentiated display on an overlapping portion of the contour of the anatomical structure with the lesion diagram to indicate that the spatial relationship between the lesion and the anatomical structure is overlapping; and
when the spatial relationship exhibits compression, performing a second differentiated display on the overlapping portion of the contour of the anatomical structure with the lesion diagram to indicate that the spatial relationship between the lesion and the anatomical structure is compressive;
wherein the first differentiated display is different from the second differentiated display.

4. The method of claim 1, further comprising:
when the determined morphological parameter comprises the relative position between the lesion diagram and the target tissue diagram, generating a lesion stalk diagram based on the relative position between the lesion diagram and the target tissue diagram, and displaying the lesion stalk diagram between the lesion diagram and the target tissue diagram; or
receiving a user-initiated operation for drawing a lesion stalk diagram between the lesion diagram and the target tissue diagram, generating the lesion stalk diagram in response to the operation, and displaying the lesion stalk diagram between the lesion diagram and the target tissue diagram;
wherein the lesion stalk diagram has a first end connected to the lesion diagram and a second end connected to the target tissue diagram.

5. The method of claim 4, wherein
the target tissue diagram of the target section comprises a contour of the anatomical structure of the target tissue, and
the second end of the lesion stalk diagram is connected to a connection point on the target tissue diagram located at a closest point on the contour of the anatomical structure adjacent to the lesion diagram, wherein a position of the connection point is adjustable on the lesion distribution diagram.

6. The method of claim 4, wherein generating the lesion stalk diagram based on the relative position between the lesion diagram and the target tissue diagram comprises:
when the lesion diagram is located outside the target tissue diagram, or when the lesion diagram is located in a cavity within the target tissue diagram, generating the lesion stalk diagram.

7. The method of claim 1, wherein, the editing operation comprises an appearance selection operation and a region selection operation; and
said determining a morphological parameter of the lesion diagram in response to an editing operation, and displaying the lesion diagram on a corresponding region of the target tissue diagram according to the morphological parameter of the lesion diagram comprises:
displaying a plurality of lesion diagrams with different appearances on the display interface for user selection, wherein the appearances represent at least one of a lesion type being benign or malignant, and a lesion contour;
taking a user-selected lesion diagram as the lesion diagram in the lesion distribution diagram in response to the appearance selection operation; and
displaying the lesion diagram in a user-selected region in response to the region selection operation.

8. The method of claim 7, further comprising:
acquiring a dimensional measurement of the lesion on an ultrasound image of the lesion; and
determining the dimension of the lesion diagram based on the dimensional measurement.

9. The method of claim 8, wherein
the dimensional measurement comprises at least one of a measured value of a length of the lesion, a measured value of a width of the lesion and a measured value of a height of the lesion; and
the plurality of lesion diagrams with different appearances comprise: a lesion diagram with an appearance representing a benign lesion, and a plurality of lesion diagrams with appearances respectively representing varying malignancy levels.

10. The method of claim 7, further comprising:
deleting a displayed lesion diagram in response to a user-initiated deletion operation on a selected lesion diagram; or
adjusting the morphological parameter of the lesion diagram in response to the user editing operation, and updating the displayed lesion diagram according to the adjusted morphological parameter.

11. The method of claim 10, wherein, said adjusting the morphological parameter of the lesion diagram in response to the user editing operation, and updating the displayed lesion diagram according to the adjusted morphological parameter comprises:
in response to a user-initiated operation for moving the displayed lesion diagram, moving the displayed lesion diagram so as to adjust the relative position between the lesion diagram and the target tissue diagram; or
in response to a user-initiated operation for stretching or scaling the displayed lesion diagram, stretching or scaling the displayed lesion diagram so as to adjust the appearance and/or dimension of the lesion diagram; or
in response to a user-initiated operation for rotating the displayed lesion diagram, rotating the displayed lesion diagram so as to adjust the orientation of the lesion diagram.

12. The method of claim 1, wherein, the display interface further displays a tumor classification guide diagram configured to prompt a user to determine a tumor type of the lesion, wherein the tumor classification guide diagram comprises a plurality of schematic graphics of different tumor types, each schematic graphic having a feature indicative of a tumor type, and different tumor types exhibiting different features; and the method further comprises:
determining the tumor type of the lesion in response to a user-initiated classification operation, and displaying a feature corresponding to the determined tumor type on the lesion diagram; or
when the determined morphological parameter comprises the relative position between the lesion diagram and the target tissue diagram, determining the tumor type of the lesion according to a predetermined classification rule based on the relative position, and displaying a feature corresponding to the determined tumor type on the lesion diagram.

13. The method of claim 1, further comprising:
based on a type of the target section, displaying, on the display interface, a patient orientation corresponding to the lesion distribution diagram of the target section; wherein, the type of the target section is one of a sagittal plane, a transverse plane, and a coronal plane, and the patient orientation comprises at least one of left, right, ventral, dorsal, cranial, and caudal orientations.

14. The method of claim 2, wherein said generating the target tissue diagram of the target section based on the ultrasound image of the target section comprises:
segmenting a contour of an anatomical structure of the target tissue from the ultrasound image of the target section, and generating the target tissue diagram of the target section based on the segmented contour of the anatomical structure of the target tissue; or
displaying a plurality of predetermined basic tissue structure diagrams of the target section on the display interface for user selection; displaying the ultrasound image of the target section on the display interface for a user to input a plurality of key points on the ultrasound image, the plurality of key points being configured to identify a position and a dimension of an anatomical structure of the target tissue diagram; and mapping positional relationships between the plurality of input key points to a user-selected basic tissue structure diagram to generate the target tissue diagram of the target section; or
displaying a plurality of predetermined basic tissue structure diagrams of the target section on the display interface for user selection; acquiring a dimensional measurement of an anatomical structure of the target tissue from the ultrasound image of the target section; and mapping the dimensional measurement of the anatomical structure to a user-selected basic tissue structure diagram to generate the target tissue diagram of the target section; or
displaying a plurality of predetermined basic tissue structure diagrams of the target section on the display interface for user selection; and taking a user-selected basic tissue structure diagram as the target tissue diagram of the target section.

15. The method of claim 1, wherein the target tissue diagram of the target section and the ultrasound image of the target section are in a substantially 1:1 proportion.

16. The method of claim 3, wherein the target tissue is a uterus, and the anatomical structure of the target tissue comprises a uterine body, an endometrium, and a cervix.

17. The method of claim 2, wherein the ultrasound image of the target section and the lesion distribution diagram are each two-dimensional images.

18. An ultrasound imaging device, comprising: an ultrasound probe, a transmitting and receiving circuit, a processor, and a human-machine interface device; wherein:
the ultrasound probe is configured to transmit ultrasound waves toward a target tissue and receive corresponding ultrasound echoes;
the transmitting and receiving circuit is configured to control the probe to transmit ultrasound waves and receive ultrasound echoes; and
the processor is configured to:
acquire a target tissue diagram of at least one target section of a target tissue, wherein the target tissue diagram of the target section is a schematic diagram for presenting a morphology of the target tissue having at least one lesion on the target section;
display the target tissue diagram of the target section on a display interface;
determine a morphological parameter of a lesion diagram of a lesion in response to an editing operation, the morphological parameter comprising at least one of an appearance of the lesion diagram, a dimension of the lesion diagram, an orientation of the lesion diagram, and a relative position between the lesion diagram and the target tissue diagram; and
display the lesion diagram on a corresponding region of the target tissue diagram according to the morphological parameter of the lesion diagram, thereby obtaining the lesion distribution diagram of the target section.

19. A computer-readable storage medium, comprising: a computer program stored thereon, the computer program being executable by a processor to:
acquire a target tissue diagram of at least one target section of a target tissue, wherein the target tissue diagram of the target section is a schematic diagram for presenting a morphology of the target tissue having at least one lesion on the target section;
display the target tissue diagram of the target section on a display interface;
determine a morphological parameter of a lesion diagram of a lesion in response to an editing operation, the morphological parameter comprising at least one of an appearance of the lesion diagram, a dimension of the lesion diagram, an orientation of the lesion diagram, and a relative position between the lesion diagram and the target tissue diagram; and
display the lesion diagram on a corresponding region of the target tissue diagram according to the morphological parameter of the lesion diagram, thereby obtaining the lesion distribution diagram of the target section.
